(19) United States
(12) Patent Application Publication
(10) Pub. No.: US 2024/0182540 A1
(43) Pub. Date: Jun. 6, 2024

(54) CHIMERIC APOPTOTIC SIGNAL TARGETING LYMPHOCYTES (TIM-4 CASTL) AND METHODS OF MAKING AND USING SAME

(71) Applicant: Duke University, Durham, NC (US)

(72) Inventors: Peter Fecci, Durham, NC (US); Daniel Wilkinson, Durham, NC (US); Ethan Srinivasan, Durham, NC (US)

(21) Appl. No.: 18/511,817

(22) Filed: Nov. 16, 2023

Related U.S. Application Data
(60) Provisional application No. 63/425,783, filed on Nov. 16, 2022.

(51) Int. Cl. C07K 14/705; A61K 39/00; A61P 35/00; C07K 14/725; C12N 5/0783

(52) U.S. Cl. CPC C07K 14/70503; A61K 39/4611; A61K 39/4637; A61K 39/464431; A61K 39/4645; A61P 35/00; C07K 14/7051; C12N 5/0638; A61K 2239/15; C07K 2319/02; C07K 2319/03; C12N 2510/00

(57) ABSTRACT

The present invention provides recombinant TIM-4 fusion proteins comprising an extracellular domain of TIM-4 and at least one co-stimulatory domain. Also provided are cells comprising the fusion protein and methods of making and using the same.

### CROSS-REFERENCE TO RELATED APPLICATIONS

This application claims priority to U.S. Provisional Application No. 63/425,783 filed on Nov. 16, 2022, the contents of which are incorporated by reference in their entireties.

### REFERENCE TO AN ELECTRONIC SEQUENCE LISTING

The contents of the electronic sequence listing (155554.00721.xml; Size: 26,939 bytes; and Date of Creation: Nov. 13, 2023) is herein incorporated by reference in its entirety.

### BACKGROUND

According to the World Health Organization, cancer is responsible for close to 10 million deaths per year worldwide. There is a critical need to advance cancer treatments beyond their current state. Immune-based therapies, such as immune checkpoint blockade (ICB) and adoptive lymphocyte transfer (ALT), have garnered excitement over the past couple of decades due to their success in certain subsets of cancers. Broadening the repertoire of cancers in which immunotherapies are efficacious remains a major goal of cancer immunology groups worldwide. ALT includes chimeric antigen receptor (CAR) T cell therapy. CAR T cells are T cells genetically modified to express an extracellular domain that recognizes an antigen of choice fused to intracellular T cell signaling components. CAR T cell therapy has achieved tremendous success in many blood cancers, but this success has failed to translate to solid tumors. At the root of failure in many solid cancers is substantial intra-tumoral heterogeneity. Many cancers possess few tumor-specific antigens and exhibit neoantigen expression profiles that vary even cell to cell. For example, the most prevalent glioblastoma (GBM) protein antigens (e.g. EGFRvIII) are present in just 30% of tumors, and then on only 30-50% of cells. This intra-tumoral heterogeneity results in antigen escape of antigen-negative tumor cell populations following CAR treatment. It has become increasingly clear that the traditional paradigm of protein-antigen targeting in certain subsets of cancer is destined for failure. Accordingly, there is a remaining need in the art for novel tumor cell targeting therapies.

### SUMMARY

The present invention provides compositions and methods for a T-cell based cancer immunotherapy comprising TIM-4, which has a high affinity for phosphatidyl serine, fused to T-cell activating machinery. One aspect of the invention provides a TIM-4 fusion protein comprising a TIM-4 extracellular domain and at least one co-stimulatory domain. In some embodiments, the TIM-4 extracellular domain comprises SEQ ID NO: 7, SEQ ID NO: 8, or a sequence having at least 95% identity to one of SEQ ID NO: 7 or 8. In some embodiments, the co-stimulatory domain comprises a portion of CD3, or further comprises one or both of a co-stimulatory domain from CD28 and 4-1BB. In some embodiments, the fusion protein further comprises a signal sequence and a transmembrane domain.

A second aspect of the disclosure provides a construct comprising a polynucleotide sequence encoding the TIM-4 fusion protein operably linked to a promoter. In some embodiments, the construct comprises a lentiviral, retroviral or AAV vector. In some embodiments, a T cell comprising the construct is provided. The T cell may express the TIM-4 fusion protein. In some embodiments, the T cell further comprises a chimeric antigen receptor (CAR), and the CAR may be specific for a tumor antigen.

Another aspect of the present disclosure provides a method of treating cancer in a subject, comprising administering a therapeutically effective amount of the T cell comprising a TIM-4 fusion protein or a polynucleotide encoding the TIM-4 fusion protein and optionally a pharmaceutically acceptable excipient, carrier and/or diluent. In some embodiments, the cancer is a PS-associated cancer or an EGFR-associated cancer. In some embodiments, the subject is administered radiation and/or chemotherapy prior to administration of the T cell, and/or administered a T cell activating immunotherapy with administration of the T cell.

### BRIEF DESCRIPTION OF THE DRAWINGS

The present technology can be better understood by reference to the following drawings. The drawings are merely exemplary to illustrate certain features that may be used singularly or in combination with other features and the present technology should not be limited to the embodiments shown.

FIG. 1. Phosphatidylserine is exposed on the surface of multiple tumor histologies and is detectable with TIM4 and is enhanced by radiation and chemotherapy. A. Murine glioma (CT-2A, SMA-560), non-glioma (B16F10, Lewis lung carcinoma), and human glioma (U87) cells were removed from culture flasks and stained with APC-conjugated recombinant mouse TIM-4. Histograms show detectable TIM-4 binding to virtually all tumor cells relative to unstained control. These data suggest TIM-4 is a viable strategy for targeting tumor cells. B. CT-2A was implanted intracranially in C57BL/6 mice. 18 days later, tumor was explanted, processed to generate a single cell suspension, then stained with APC-conjugated recombinant TIM4. C. CT2A Tumor cells were exposed to 10 Gy radiation, plated for 24 hours, and then stained with TIM4-APC. D, E. C57BL/6 mice bearing intracranial CT2A-GFP underwent 10 Gy total body irradiation 15 days after tumor implantation. 48 hours later, tumors were excised and stained with TIM4-APC. Tumor cells were gated on GFP⁺ cells prior to analysis for TIM4-APC binding. F. CT2A tumor cells were cultured in the presence of 1000 μM temozolomide (TMZ). After 24 hours, cells were stained with TIM4-APC.

FIG. 2. TIM4 CASTL redirects T cells to kill tumor cells. A. Design of third generation TIM4 CASTL. B. Schematic showing the difference between a traditional CAR and the TIM4 CASTL. C. Transduction efficiency of TIM4 CASTL as shown by anti-TIM4 binding 72 hours post-transduction. D-F. In vitro cytotoxicity against CT2A (D), SMA-560 (E), and B16F10 (F). G. In vitro cytotoxicity of VIII CAR, TIM4 CASTL, or a 50:50 mix of VIII CAR and TIM4 CASTL against a 70:30 mix of CT2A:CT2AvIII. H. In vitro cytotoxicity of VIII CAR and TIM4 CASTL against CT2A that underwent exposure to 10 Gy radiation 24 hours prior to co-culture or were non-irradiated. I. In vitro cytotoxicity of wildtype TIM4 CASTL and the TIM4 mutant CASTL against CT2A.

FIG. 3. TIM4 CASTL is effective in vivo under certain circumstances. A. CT2A glioma was implanted intracranially in C57BL/6 mice. 5 days later, mice were administered 400 mg/kg TMZ i.p.to deplete lymphocytes. 24 hours later (day 6), 2×10⁶ Nontransduced (NT) T cells or TIM4 CASTL were administered IC at the tumor site. B. CT2A glioma was implanted intracranially in C57BL/6 mice. 5 days later, mice were administered 400 mg/kg TMZ i.p. to deplete lymphocytes. 24 hours later (day 6), 2×10⁶ Nontransduced (NT) T cells or 2^{nd} generation TIM4-CD28 CASTL were administered IC at the tumor site. C. CT2A glioma was implanted intracranially in C57BL/6 mice. 5 days later, mice were administered 400 mg/kg TMZ i.p. to deplete lymphocytes. 24 hours later (day 6), 2×10⁶ Nontransduced (NT) T cells, 3^{rd} generation TIM4 CASTL, or 2^{nd} generation TIM4-CD28 CASTL were administered IC at the tumor site. 200 μg agonist 4-1BB antibody was administered i.p. to all mice on days 6, 9, and 12. D. CT2A glioma was implanted intracranially in C57BL/6 mice. 5 days later, mice were administered 400 mg/kg TMZ i.p. to deplete lymphocytes. 24 hours later (day 6), 2×10⁶ Nontransduced (NT) T cells, 3^{rd} generation TIM4 CASTL (TIM4wt), or TIM4 mutant CASTL (TIM4mut) were administered IC at the tumor site. 200 μg agonist 4-1BB antibody was administered i.p. to all mice on days 6, 9, and 12.

### DETAILED DESCRIPTION

The present invention provides compositions and methods for a T-cell based immunotherapy for cancer. This novel therapy employs TIM-4, which has a high affinity for phosphatidyl serine, connected to T-cell activating machinery. Unlike CAR T cell technology, this disclosure comprises a transduced construct comprising a natural receptor which targets cell-surface exposed phosphatidyl serine (PS).

The inventors provide herein a TIM-4 cytotoxic T cell (termed TIM-4 chimeric apoptotic signal targeting lymphocyte [CASTL, “castle’]) and methods of making and using same. Data provided herein reveals that TIM-4 cytotoxic T cells selectively bind multiple tumor lines in homogeneous fashion, binding that is further enhanced following radiation, a standard of care cancer treatment. Further, the TIM-4 transduced cytotoxic T cells provided herein target PS on tumor cells and elicit tumor cell killing. The inventors also demonstrate the use of a mutant TIM-4 extracellular domain and also demonstrate the combination of TIM-4 CASTL with CAR T-cells. Finally, the inventors demonstrate the unexpected efficacy of the TIM-4 CASTL with alternative intracellular signaling domain configurations and with the addition of a soluble T cell activating immunotherapy.

### Fusion Proteins and Constructs

One aspect of the present invention provides a recombinant TIM-4 fusion protein comprising a TIM-4 extracellular domain and at least one co-stimulatory domain. As used herein, a “fusion protein” may also be called a chimeric protein and are proteins created through the joining of two or more genes or portions thereof that originally encoded for separate proteins. Translation of the fusion gene results in a single or multiple polypeptides with functional properties derived from each of the original proteins. Fusion proteins may occur in the body by transfer of DNA between chromosomes or be made recombinantly by combining genes or parts of genes such that a single transcript is formed encoding both polypeptides in frame from the same or different organisms.

The fusion protein of the present invention may comprise an extracellular domain with at least one antigen specific targeting region, a transmembrane domain, and an intracellular domain including one or more co-stimulatory domains in a combination that is not naturally found together on a single protein. This particularly includes receptors wherein the extracellular domain and the cytoplasmic domain are not naturally found together on a single receptor protein. An extracellular domain is external to the cell or organelle and functions to recognize and respond to a ligand. A transmembrane domain spans the membrane of a cell, and an intracellular domain is situated inside a cell. Intracellular co-stimulatory domains provide secondary signals to the cell. They can recruit signaling molecules, cytoskeletal mobilization or induce cell proliferation, differentiation or survival.

T cell membrane protein 4, or TIM-4 (also may be known as T cell membrane protein 4 or T-cell immunoglobulin and mucin domain containing 4, TIM4) is a protein in humans that is encoded by the TIMD4 gene. Tim-4 is a phosphatidylserine (PS) receptor that is expressed on various immune cell and macrophage subsets. PS is a phospholipid typically present on the cytoplasm-facing side of the plasma membrane, where it remains out of view to the immune system. Cellular stresses can lead to dysregulation of processes that keep PS facing internally and instead promote PS exposure on the cell surface. Tumor cells frequently lose the capacity to regulate their plasma membrane and exhibit detectable levels of PS on their surface, as may normal cells undergoing apoptosis. PS can have a tolerizing effect on the immune system when exposed on the cell surface. While PS exposure thus can be tumor-adaptive, it also leaves the tumor with an “Achilles Heel” that can be targeted immunologically or otherwise. For instance, TIM family proteins bind PS with varying affinity, the strongest binder being TIM-4.

The recombinant TIM-4 fusion protein of the present disclosure may comprise a TIM-4 extracellular domain comprising SEQ ID NO: 7 or a sequence having at least 95% identity to SEQ ID NO: 7 and capable of binding to PS. The TIM-4 fusion protein may also comprise a mutant TIM-4 extracellular domain, wherein the domain comprises mutations which alter the binding affinity of TIM-4 for PS. For example, the TIM-4 fusion protein may comprise a TIM-4 extracellular domain comprising SEQ ID NO: 8 or a sequence having at least 95% identity to SEQ ID NO: 8 and capable of binding to PS, wherein the residues WFN at positions 95-97 of SEQ ID NO: 7 are mutated to AAA residues (see Santiago et al. Immunity, V. 27, 941-951 2007).

The TIM-4 fusion protein may further comprise an N-terminal signal sequence. The N-terminal signal sequence may comprise a secretion signal sequence. A secretion signal sequence or peptide comprises a short peptide present at the N-terminus of proteins that are destined toward the secretory or cell surface expression pathway. These proteins include those that reside either inside certain organelles, secreted from the cell, or inserted into cellular membranes. Signal sequences may be removed or cleaved off from the fusion protein during cellular processing of the protein. Thus, when administered as a protein, the TIM-4 fusion protein will generally lack the signal sequence, but the signal sequence may be needed to allow for adequate expression and secretion or cell surface expression of the protein. In some embodiments, the secretion signal sequence includes a GMCSFR signal sequence, including SEQ ID NO: 9 or sequences with at least 90% identity to SEQ ID NO: 9. Signal sequences are generally 15-22 amino acids in length and those of skill in the art will appreciate that one signal sequence can be removed, and another signal sequence used in its place.

The fusion protein of the present disclosure may comprise a transmembrane and hinge sequence. A hinge sequence is a short sequence of amino acids that facilitates antibody flexibility (see, e.g., Woof et al., Nat. Rev. Immunol., 4(2): 89-99 (2004)). The hinge sequence may be positioned between the antigen recognition or binding moiety (e.g. TIM-4) and the transmembrane domain. The hinge sequence can be any suitable sequence derived or obtained from any suitable molecule. In some embodiments, for example, the hinge sequence is derived from a CD8a molecule or a CD28 molecule. A suitable hinge and transmembrane region can be found in SEQ ID NO: 10 or a sequence having at least 90% identity to SEQ ID NO: 10.

The transmembrane domain may be derived either from a natural or from a synthetic source. Where the source is natural, the domain may be derived from any membrane-bound or transmembrane protein. Transmembrane regions of particular use in this invention may be derived from (i.e., comprise at least the transmembrane region(s) of) the a, b, d, or g chain of the T-cell receptor, CD28, CD3.epsilon., CD3z, CD45, CD4, CD5, CD8. CD9, CD16, CD22, CD33, CD37, CD64, CD80, CD86, CD134, CD137, or CD154. For example, the hinge and transmembrane used herein comprises the CD8 hinge and transmembrane region of SEQ ID NO: 10. In some embodiments, the transmembrane domain may be synthetic, in which case it may comprise predominantly hydrophobic residues such as leucine and valine. In some embodiments, a triplet of phenylalanine, tryptophan and valine may be found at each end of a synthetic transmembrane domain. In some embodiments, a short oligo- or polypeptide linker, having a length of, for example, between about 2 and about 10 (such as about any of 2, 3, 4, 5, 6, 7, 8, 9, or 10) amino acids in length may form the linkage between the transmembrane domain and the extracellular domain of the fusion protein as provided herein.

The fusion protein of the present disclosure may comprise at least one intracellular signaling domain, region or co-stimulatory molecule. The intracellular signaling domain may be a co-stimulatory domain. A costimulatory domain is required for an efficient antigen response in immune cells. The intracellular signaling domain of the TIM-4 fusion protein provided herein is responsible for activation of at least one of the normal effector functions of the cell in which the TIM-4 fusion protein as provided herein has been placed. For example, effector function of a T cell, may be cytolytic activity or helper activity including the secretion of cytokines. Thus, the term “intracellular signaling domain” refers to the portion of a protein which transduces the effector function signal and directs the cell to perform a specialized function. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term “intracellular signaling sequence” is thus meant to include any truncated portion of the intracellular signaling domain sufficient to transduce the effector function signal.

In particular embodiments, the intracellular signaling domain is derived from CD3 zeta (CD3ζ (TCR zeta, GenBank acc. no. BAG36664.1). T-cell glycoprotein CD3 zeta (CD3ζ chain, also known as T-cell receptor T3 zeta chain or CD247 (Cluster of Differentiation 247), is a protein that in humans is encoded by the CD247 gene. The fusion protein of the present invention may also optionally comprise additional co-stimulatory domains, including CD28, 4-1BB, OX-40, ICOS or other members of the TNF receptor superfamily or immunoglobulin (Ig) superfamily. Members of the TNF superfamily form trimeric structures, and their monomers are composed of beta-strands that orient themselves into a two-sheet structure. The TNF superfamily ligands include lymphotoxin alpha, tumor necrosis factor, lymphotoxin beta, OX40 ligand, CD40 ligand, Fas ligand, CD27 ligand, CD30 ligand, CD137 ligand, TNF-related apoptosis-inducing ligand, receptor activator of nuclear factor kappa-B ligand, TNF-related weak inducer of apoptosis, a proliferation-inducing ligand, B-cell activating factor, LIGHT, vascular endothelial growth factor, TNF superfamily member 18 and ectodysplasin A. These ligands then bind to receptors in the TNF superfamily. Ig superfamily members are characterized based on shared structural features with immunoglobulins (aka antibodies), including an immunoglobulin domain with a characteristic Ig-fold. The Ig domain is reported to be one of the most populous family of proteins in the human genome with over 700 members identified and known in the art. Co-stimulatory domains of the present disclosure may comprise CD3ζ of SEQ ID NO: 13, CD28 of SEQ ID NO: 11, or 4-1BB of SEQ ID NO:12 or sequences with at least 95% identity to these. These co-stimulatory domains may be used in isolation or in any combination or order. In particular embodiments, CD3ζ and CD28 are used without 4-1BB.

While the fusion protein of the present disclosure is exemplified with the above mentioned co-stimulatory molecules, other co-stimulatory domains, including CD27, CD30, CD40, ICOS, lymphocyte function-associated antigen-1 (LFA-1), CD2, CD7, LIGHT, NKG2C, B7-H3, and a ligand that specifically binds with CD83, CD8, CD4, b2c, CD80, CD86, DAP10, DAP12, MyD88, BTNL3, and NKG2 can be used alone or in combination with other co-stimulatory molecules.

By way of example, but not by way of limitation, in some embodiments, the disclosed TIM-4 fusion protein may comprise a signal sequence, a mutated or wild type TIM-4 extracellular domain, a CD8 hinge and transmembrane domain, and a CD28, 4-1BB and CD3ζ intracellular signaling domain (SEQ ID NO: 3 or 4 or sequences having at least 95% identity to these sequences). Alternatively, the TIM-4 fusion protein may comprise a signal sequence, a mutated or wild type TIM-4 extracellular domain, a CD8 hinge and transmembrane domain, and a CD28 and CD3ζ intracellular signaling domain (SEQ ID NO: 5 or 6 or sequences having at least 95% identity to these sequences).

Some embodiments of the present disclosure describe a construct comprising a heterologous promoter operably connected to a polynucleotide encoding a TIM-4 fusion protein described herein. The term “construct” or “polynucleotide construct” is a polynucleotide which allows the encoded sequence to be replicated and/or expressed in the target cell. A construct may contain an exogenous promoter, operably linked to any one of the polynucleotides described herein. As used herein, a polynucleotide is “operably connected” or “operably linked” when it is placed into a functional relationship with a second polynucleotide sequence. As used herein, the terms “heterologous promoter,” “promoter,” “promoter region,” or “promoter sequence” refer generally to transcriptional regulatory regions of a gene, which may be found at the 5′ or 3′ side of a polynucleotides described herein, or within the coding region of said polynucleotides. Typically, a promoter is a DNA regulatory region capable of binding RNA polymerase in a cell and initiating transcription of a downstream (3′ direction) coding sequence. The typical 5′ promoter sequence is bounded at its 3′ terminus by the transcription initiation site and extends upstream (5′ direction) to include the minimum number of bases or elements necessary to initiate transcription at levels detectable above background. Within the promoter sequence is a transcription initiation site (conveniently defined by mapping with nuclease S1), as well as protein binding domains (consensus sequences) responsible for the binding of RNA polymerase.

The term “polynucleotide” is used herein interchangeably with the term “nucleic acid” and refers to an organic polymer composed of two or more monomers including nucleotides, nucleosides or analogs thereof, including but not limited to single stranded or double stranded, sense or antisense deoxyribonucleic acid (DNA) of any length and, where appropriate, single stranded or double stranded, sense or antisense ribonucleic acid (RNA) of any length, including siRNA. The term “nucleotide” refers to any of several compounds that consist of a ribose or deoxyribose sugar joined to a purine or a pyrimidine base and to a phosphate group, and that are the basic structural units of nucleic acids. The term “nucleoside” refers to a compound (as guanosine or adenosine) that consists of a purine or pyrimidine base combined with deoxyribose or ribose and is found especially in nucleic acids. The term “nucleotide analog” or “nucleoside analog” refers, respectively, to a nucleotide or nucleoside in which one or more individual atoms have been replaced with a different atom or with a different functional group. Accordingly, the term polynucleotide includes nucleic acids of any length, including DNA, RNA, ORFs, analogs and fragments thereof.

The polynucleotides and constructs described herein encode and allow the expression of polypeptides or proteins. Polypeptides and proteins are used interchangeably here in and refers to a polymer composed of amino acids linked via peptide bonds. As noted above the fusion proteins provided here are provided as shorter polypeptide segments from other naturally occurring proteins. The sequences provided herein can be linked via peptide linkers or linked directly to each other via a single peptide bond. Linkers may be 1-20 amino acids in length and may comprise any amino acid sequence. Linkers commonly used in the art include glycine and serine containing linkers as these amino acids are neutral and contain small side-chains. The polypeptides provided herein may also contain mutations that may or may not affect the function of the fusion protein. The TIM4 fusion protein may have less than 100% sequence identity with those fusion proteins provided here and maintain the ability to bind to PS on the surface of a target cell and send a co-stimulatory signal to effect a cellular response. Thus, fusion proteins or portions thereof with at least 85%, 90%, 92%, 94%, 95%, 96%, 97%, 98%, 99% identity to the TIM4 fusion proteins provided are also encompassed herein.

In some embodiments, the construct is an expression construct, a vector or a viral vector. A vector is any particle used as a vehicle to artificially carry a foreign nucleic sequence, typically DNA, into another cell, where it can be replicated and/or expressed. A vector containing foreign DNA is termed recombinant DNA. The four major types of vectors are plasmids, viral vectors, cosmids, and artificial chromosomes. Expression constructs comprise a heterologous promoter and the nucleic acid sequence encoding protein of interest (e.g., TIM-4) which is capable of expression in the cell in which it is introduced. The expression constructs include vectors which are capable of directing the expression of exogenous genes to which they are operatively linked. Such vectors are referred to herein as “recombinant constructs,” “expression constructs,” “recombinant expression vectors” (or simply, “expression vectors” or “vectors”) and may be used interchangeably. Suitable vectors are known in the art and contain the necessary elements in order for the gene encoded within the vector to be expressed as a protein in the host cell. The term “vector” refers to a nucleic acid molecule capable of transporting another nucleic acid to which it has been linked. One type of vector is a “plasmid”, which refers to a circular double stranded DNA loop into which additional DNA segments may be ligated, specifically exogenous DNA segments. Another type of vector is a viral vector, wherein additional DNA segments may be ligated into the viral genome. Viral vectors are incorporated into viral particles that are then used to transport the viral polynucleotide encoding the protein of interest into the target cells. Certain vectors are capable of autonomous replication in a host cell into which they are introduced. Other vectors can be integrated into the genome of a host cell upon introduction into the host cell, and thereby are replicated along with the host genome (e.g., lentiviral vectors). Moreover, certain vectors are capable of directing the expression of exogenous genes to which they are operatively linked. In general, vectors of utility in recombinant DNA techniques are often in the form of plasmids. In the present specification “vector” includes expression vectors, such as viral vectors (e.g., replication defective retroviruses (including lentiviruses), adenoviruses and adeno-associated viruses (AAV)), which serve equivalent functions.

The vectors are heterogeneous exogenous constructs containing sequences from two or more different sources. Suitable vectors include, but are not limited to, plasmids, expression vectors, lentiviruses (lentiviral vectors), adeno-associated viral vectors (rAAV), among others and includes constructs that are able to express the protein of interest. A preferred vector is a lentiviral vector, retroviral vector or adeno-associated vector. Suitable methods of making viral particles are known in the art to be able to transform cells in order to express the protein of interest described herein.

Heterologous promoters useful in the practice of the present invention include, but are not limited to, constitutive, inducible, temporally-regulated, developmentally regulated, chemically regulated, tissue-preferred, tissue-specific promoters and cell-type specific. The heterologous promoter may be a plant, animal, bacterial, fungal, or synthetic promoter. Suitable promoters are known and described in the art. In mammalian cells, typical promoters include, without limitation, promoters for Rous sarcoma virus (RSV), human immunodeficiency virus (HIV-1), cytomegalovirus (CMV), SV40 virus, as well as the translational elongation factor EF-1α promoter or ubiquitin promoter.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York). In some embodiments, the introduction of a polynucleotide into a host cell is carried out by calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus 1, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Regardless of the method used to introduce exogenous nucleic acids into a host cell or otherwise expose a cell to the inhibitor of the present invention, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays. In some embodiments, detection is facilitated by the inclusion of a reporter gene or tag within the recombinant sequence. Suitable reporter genes may include genes encoding luciferase, β-galactosidase, chloramphenicol acetyl transferase, secreted alkaline phosphatase, or the green fluorescent protein gene (e.g., Ui-Tel et al., FEBS Letters, 479, 2000). A tag may also be used for purification, tracking or imaging the fusion protein.

In some embodiments, sequence variants provided herein are contemplated. For example, it may be desirable to alter the binding affinity and/or other biological properties of TIM-4. For example, SEQ ID NO: 8 is a mutated TIM-4 extracellular domain sequence, which lowers the binding affinity of TIM-4 for PS. Amino acid sequence variants of a fusion protein or construct may be prepared by introducing appropriate modifications into the nucleotide sequence encoding the fusion protein, or by peptide synthesis. Such modifications include, for example, deletions from, and/or insertions into and/or substitutions of residues within the amino acid sequences of the antibody moiety. Any combination of deletion, insertion, and substitution can be made to arrive at the final construct, provided that the final construct possesses the desired characteristics, e.g., antigen- or ligand-binding. Compositions and sequences provided herein, including recombinant TIM-4 fusion proteins may also comprise species specific variations. For example, SEQ ID NOs: 1, 2, are murine sequences and SEQ ID NOs: 3-13 are human sequences. Species specific sequences and variations may be used depending on the desired subject species and outcomes.

In some embodiments, the constructs encoding a fusion protein described herein are introduced into a cell. The cell may be of any type wherein the expression of the TIM-4 fusion protein is desired. In some embodiments, the cell may be an immune cell, in particular the cell may be a T lymphocyte.

In some embodiments, the T cell may additionally comprise a chimeric antigen receptor. The term “chimeric antigen receptor” or “chimeric receptor” or “CAR” or “CARs” as used herein refers to a polypeptide having a pre-defined binding specificity to a desired target and operably connected to (e.g., as a fusion or as separate chains linked by one or more disulfide bonds, etc.) the intracellular part of a T-cell activation domain. More particularly, CAR are engineered receptors, which, when expressed graft an antigen specificity onto a cytotoxic cell, for example T cells, NK cells or macrophages. Suitably, CAR proteins are engineered to give T cells the new ability to target a specific protein. The CARs of the present invention may comprise an extracellular domain with at least one antigen specific targeting region, a transmembrane domain and an intracellular domain including one or more co-stimulatory domains in a combination that is not naturally found together on a single protein. This particularly includes receptors wherein the extracellular domain and the cytoplasmic domain are not naturally found together on a single receptor protein. Further, the chimeric receptor is different from the TCR expressed in the native T cell lymphocyte.

The CAR of the present disclosure may be of any specificity. In some embodiments, the CAR may be specific for a tumor associated or tumor specific antigen. For example, a CAR targeting HER2, B7-H3 or mesothelin (MSLN) may be used. A CAR may localize the T cell to a specific location or target. For example, EGFR (and EGFR vIII) is expressed in some tumors and is considered a tumor specific antigen in glioblastoma tumors. A T cell of the present disclosure may comprise a CAR-T cell specific for EGFR and/or EGFR vIII, as well as the TIM-4 fusion protein.

### Methods

The present invention also provides a method of treating cancer in a subject, comprising administering a therapeutically effective amount of the T cell comprising a construct encoding a TIM-4 fusion protein described herein. The method may also comprise administering a therapeutically effective amount of the CAR-T cell comprising a construct encoding a TIM-4 fusion protein described herein, wherein the CAR is specific for a tumor antigen.

The term “subject” may be used interchangeably with the terms “individual” and “patient” and includes human and non-human mammalian subjects. In some embodiments, the subject is human. A subject as utilized herein may refer to a subject in need of treatment for a disease or disorder associated with a suspected tumor or cancer. A subject in need thereof may include a subject having a cancer that is characterized by gross abnormality visible by X-ray, computerized tomography (CT), or magnetic resonance imaging (MRI).

Treating cancer in a subject includes the reducing, repressing, delaying or preventing cancer growth, reduction of tumor volume, and/or preventing, repressing, delaying or reducing metastasis of the tumor. Treating cancer in a subject also includes the reduction of the number of tumor cells within the subject. The term “treatment” can be characterized by at least one of the following: (a) reducing, slowing or inhibiting growth of cancer and cancer cells, including slowing or inhibiting the growth of metastatic cancer cells; (b) preventing further growth of tumors; (c) reducing or preventing metastasis of cancer cells within a subject; and (d) reducing or ameliorating at least one symptom of cancer. In some embodiments, the optimum effective amount can be readily determined by one skilled in the art using routine experimentation. The aim of treatment includes the alleviation or prevention of symptoms, slowing or stopping the progression or worsening of a disease, disorder, or condition and/or the remission of the disease, disorder or condition. The term “effective amount” or “therapeutically effective amount” refers to an amount sufficient to effect beneficial or desirable biological and/or clinical results.

As used herein, the term “administering” an agent, such as a therapeutic entity to composition described herein an animal or cell, is intended to refer to dispensing, delivering or applying the substance to the intended target. In terms of the therapeutic agent composition, the term “administering” is intended to refer to contacting or dispensing, delivering or applying the therapeutic agent to a subject by any suitable route for delivery of the therapeutic agent to the desired location in the animal, including delivery by either the parenteral or oral route, intramuscular injection, subcutaneous/intradermal injection, intravenous injection, intrathecal administration, intratumoral administration, buccal administration, transdermal delivery, topical administration, and administration by the intranasal or respiratory tract route.

The methods of the present disclosure can be used to treat any cancer, and any metastases thereof, including, but not limited to, carcinoma, lymphoma, blastoma, sarcoma, and leukemia. In some embodiments, the cancer is characterized by, or associated with phosphatidylserine (PS) expression (herein referred to as an “PS-associated cancer”). PS is a phospholipid that is normally present on the inner leaflet of normal cells. However, apoptotic as well as non-apoptotic cancer cells such as malignant melanoma, leukemia, neuroblastoma, and gastric carcinoma have been shown to widely express PS on their surfaces. PS exposed on the surface of tumor cells contributes to suppression of T-cell activity and blocks tumor clearance. In some embodiments, the cancer comprises those with PS on the cell surface. In some embodiments the cancer comprises EGFR-associated cancers which are those cancer associated with EGFR expression. Suitable examples include, but are not limited to, of such cancers include breast cancer, prostate cancer, colon cancer, squamous cell cancer, small-cell lung cancer, non-small cell lung cancer, ovarian cancer, cervical cancer, gastrointestinal cancer, pancreatic cancer, glioblastoma, liver cancer, bladder cancer, hepatoma, colorectal cancer, uterine cervical cancer, endometrial carcinoma, salivary gland carcinoma, mesothelioma, kidney cancer, vulval cancer, pancreatic cancer, thyroid cancer, hepatic carcinoma, skin cancer, melanoma, brain cancer, neuroblastoma, myeloma, various types of head and neck cancer, acute lymphoblastic leukemia, acute myeloid leukemia, Ewing sarcoma and peripheral neuroepithelioma. In some embodiments, the cancer comprises a glioma, glioblastoma, medulloblastoma, ependymoma, diffuse intrinsic pontine glioma (DIPG), a brain metastases, head and neck, ovarian, cervical, bladder or esophageal cancer

In some embodiments, the T cell comprising a construct encoding a TIM-4 fusion protein may further comprise a pharmaceutically acceptable excipient, carrier, and/or diluent. As used herein, the term “carrier” refers to a pharmaceutically acceptable solid or liquid filler, diluent or encapsulating material. A water-containing liquid carrier can contain pharmaceutically acceptable additives such as acidifying agents, alkalizing agents, antimicrobial preservatives, antioxidants, buffering agents, chelating agents, complexing agents, solubilizing agents, humectants, solvents, suspending and/or viscosity-increasing agents, tonicity agents, wetting agents or other biocompatible materials. A tabulation of ingredients listed by the above categories, may be found in the U.S. Pharmacopeia National Formulary, 1857-1859, (1990).

Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservative.

Some examples of the materials which can serve as pharmaceutically acceptable carriers are sugars, such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols, such as propylene glycol; polyols such as glycerin, sorbitol, mannitol and polyethylene glycol; esters such as ethyl oleate and ethyl laurate; agar; buffering agents such as magnesium hydroxide and aluminum hydroxide; alginic acid; pyrogen free water; isotonic saline; Ringer's solution, ethyl alcohol and phosphate buffer solutions, as well as other nontoxic compatible substances used in pharmaceutical formulations. Wetting agents, emulsifiers and lubricants such as sodium lauryl sulfate and magnesium stearate, as well as coloring agents, release agents, coating agents, sweetening, flavoring and perfuming agents, preservatives and antioxidants can also be present in the compositions, according to the desires of the formulator.

Examples of pharmaceutically acceptable antioxidants include water soluble antioxidants such as ascorbic acid, cysteine hydrochloride, sodium bisulfate, sodium metabisulfite, sodium sulfite and the like; oil-soluble antioxidants such as ascorbyl palmitate, butylated hydroxyanisole (BHA), butylated hydroxytoluene (BHT), lecithin, propyl gallate, alpha-tocopherol and the like; and metal-chelating agents such as citric acid, ethylenediamine tetraacetic acid (EDTA), sorbitol, tartaric acid, phosphoric acid and the like.

The present formulation may also comprise other suitable agents such as a stabilizing delivery vehicle, carrier, support or complex-forming species. Additionally, biologically acceptable buffer to maintain a pH close to neutral (e.g., 7.0-7.3). The coordinate administration methods and combinatorial formulations of the instant invention may optionally incorporate effective carriers, processing agents, or delivery vehicles, to provide improved formulations for delivery of the fusion protein, or cell comprising the fusion protein described herein.

The compositions disclosed herein may also be co-administered with additional therapies. Co-administration refers to administration at the same time in an individual, and also may include administrations that are spaced by hours, days, weeks, or longer, as long as the administration of multiple therapeutic agents is the result of a single treatment plan. By way of example, and not limitation, the T cell comprising a construct encoding a TIM-4 fusion protein may be co-administered with CAR-T cells or with one or more additional therapeutic agents. Such agents may include, but are not limited to, chemotherapeutic agents, radiation, checkpoint inhibitor therapy, small molecule drugs, monoclonal antibodies, antibody-drug conjugates, immunotherapy, and the like.

In an exemplary treatment schedule, a subject may undergo a treatment which induces cellular stress, then T cell comprising a construct encoding a TIM-4 fusion protein or encoding a TIM-4 fusion protein and a CAR may be administered along with a soluble T cell activating immunotherapy. A treatment which induces cellular stress may include but is not limited to administration of a chemotherapeutic agent and/or radiation. A T cell activating immunotherapy may include but is not limited to immune checkpoint inhibitors (PD-1, PDL-1, CTLA-4), monoclonal antibodies, cytokines (interferons, interleukins) and costimulatory targets such as glucocorticoid-induced tumor necrosis factor receptor (GITR), OX40, 4-1BB, CD40, and CD28).

Some embodiments of the present disclosure provide a method of treating cancer comprising administering a therapeutically effective amount of a T cell comprising a construct encoding a TIM-4 fusion protein wherein the administration is intratumoral, intracranial or intrathecal. These routes of administration have the advantage of proper trafficking of the T cells directly to the tumor or to the CNS and avoiding leakage into the periphery. Intratumoral and intracranial injections of the T cells may occur after surgery, for example in a resection cavity.

Some embodiments of the present disclosure provide a method of treating cancer comprising administering a therapeutically effective amount of a T cell comprising a construct encoding a TIM-4 fusion protein and a CAR wherein the administration is intratumoral or systemic.

The compositions described herein will generally be used in an amount effective to achieve the intended result, for example in an amount effective to treat or prevent cancer. By therapeutic benefit is meant eradication or amelioration of the underlying disorder being treated and/or eradication or amelioration of one or more of the symptoms associated with the underlying disorder such that the patient reports an improvement in feeling or condition, notwithstanding that the patient may still be afflicted with the underlying disorder. Therapeutic benefit also generally includes halting or slowing the progression of the disease, regardless of whether improvement is realized.

The amount of composition administered will depend upon a variety of factors, including, for example, the particular indication being treated, the mode of administration, whether the desired benefit is prophylactic or therapeutic, the severity of the indication being treated and the age and weight of the patient, the bioavailability of the particular composition, the conversion rate and efficiency of delivery under the selected route of administration, etc. Determination of an effective dosage for a particular use and mode of administration is well within the capabilities of those skilled in the art. Effective dosages may be estimated initially from in vitro activity and metabolism assays and/or from in vivo data, such as animal models.

Dosage amount and interval may be adjusted individually to provide levels which are sufficient to maintain therapeutic or prophylactic effect. For example, the compositions may be administered once per week, several times per week (e.g., every other day), once per day or multiple times per day, depending upon, among other things, the mode of administration, the specific indication being treated and the judgment of the prescribing physician.

### Additional Definitions

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps.

All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter.

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

In those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

The following examples are meant only to be illustrative and are not meant as limitations on the scope of the invention or of the appended claims.

### EXAMPLES

### Example 1

In the following example, the inventors describe a novel TIM-4 cytotoxic T cell (termed TIM-4 chimeric apoptotic signal targeting lymphocyte [CASTL, “castle’]) and methods of making and using same. Data provided herein reveal that TIM-4 cytotoxic T cells selectively bind multiple murine tumor lines including melanoma, glioma and lung cancer cell. The inventors demonstrate that this binding is further enhanced following radiation and that TIM-4 CASTL kills tumor cells and provides a survival benefit to the subject. In addition, the inventors create a mutant TIM-4 CASTL as well as a TIM-4 CASTL which only includes CD28 and CD3 co-stimulatory domains. Interestingly, these variations, improve tumor cell killing and survival. These compositions can be further combined with a T cell activating therapy, such as a 4-1BB agonist to further improve survival.

### Materials and Methods

### Cell Lines

Cell lines examined in this study included murine glioma lines CT2A, CT2A-GFP, and SMA-560, murine melanoma line B16F10, murine Lewis lung carcinoma (LLC), and human glioma line U87. Additionally, CT2A was genetically modified to express the EGFRvIII variant of EGFR, generating CT2AvII. CT2A, B16F10, and LLC are syngeneic on the C57BL/6 background, while SMA-560 is syngeneic on the VM/Dk background. All cell lines were cultured in DMEM+10% FBS.

### Phosphatidylserine Binding and Flow Cytometry

Recombinant Fc-tagged human TIM4 (R&D Systems) was conjugated to allophycocyanin (APC) using an APC conjugation kit (Abcam: ab201807) to generate TIM4-APC. For phosphatidylserine binding analysis of in vitro cells, cultured tumor cells were harvested from flasks using 0.05% trypsin/EDTA and resuspended in Annexin V Binding Buffer (BioLegend). Cells were then incubated with TIM4-APC and/or Annexin V-FITC for 20 minutes at room temperature followed by extensive washing in Annexin V Binding Buffer. For ex vivo binding analysis, CT2A-GFP glioma tumors were harvested from mice and processed into a single-cell suspension prior to TIM4-APC staining. Analysis was performed on GFP⁺ tumor cells. When indicated, APC-conjugated recombinant human CD19 was used as a negative staining control. To test the effect of radiation on PS exposure and TIM4 binding in vitro, tumor cells were exposed to Gy gamma irradiation and then allowed to recover for 24 hours. Cells were then analyzed for TIM4 binding. To test the effect of radiation on PS exposure in vivo, intracranial CT2A-GFP tumor-bearing mice were exposed to 10 Gy total body irradiation at day 15 post-tumor implantation. 48 hours later, tumors were excised and stained with TIM4-APC. To test the effect of temozolomide (TMZ) chemotherapy on PS exposure in vitro, tumor cells were cultured in the presence of 1000 μM TMZ for 24 hours prior to TIM4-APC staining.

The following antibody clones were used for each target: mouse CD3 (2C11; BioLegend), mouse CD8 (53-6.7; BioLegend), and mouse TIM4 (RMT4-54; BioLegend). Samples were acquired on an LSRFortessa (BD Biosciences) and analyzed using FlowJo Version 10 (BD Biosciences).

### TIM4 CASTL and VIII CAR Constructs

All CASTL and CAR constructs incorporated a second- or third-generation design, as indicated. For third-generation TIM4 CASTL generation, the extracellular (EC) domain of murine TIM4 was fused to a CD8 hinge and transmembrane (TM) domain, a CD28 costimulatory domain, a 4-1BB costimulatory domain, and a CD3ζ signaling domain. A second-generation construct consisted of the TIM4 EC domain, CD8 hinge and TM domain, CD28 domain, and CD3ζ domain. A third-generation EGFRvIII-targeting CAR (VIII CAR) consisted of an anti-EGFRvIIIscFv (clone 139) fused to CD8 hinge and TM, CD28, 4-1BB, and CD3ζ. A mutated TIM4 CASTL was also generated by mutating the three amino acid PS-binding site of TIM4 (residues 119-121; WFN) to a triple alanine repeat (AAA).

### CAR and CASTL T Cell Production

We engineered T cells to express the second- or third-generation TIM4 CASTL or VIII CAR by retroviral transduction. Briefly, HEK293T cells were transfected with vectors encoding TIM4 CASTL or VIII CAR and vectors encoding retroviral packaging genes to generate CASTL and CAR-encoding retrovirus. Retrovirus was then used to transduce mouse T cells 48 hours post-activation. Mouse T cells were activated from spleens of C57BL/6 mice by stimulation with concanavalin A in the presence of 50 U/mL human IL-2. T cells were split every 1-2 days in T cell media (RPMI supplemented with 10% FBS, non-essential amino acids, L-glutamine, sodium pyruvate, penicillin-streptomycin, gentamycin, and (3-mercaptoethanol) containing 50 U/mL human IL-2.

### In Vitro Cytotoxicity

In vitro CASTL and CAR cytotoxicity assays were performed by co-culturing T cells (effector; E) with tumor cells (target; T) at the designated E:T ratio. Before co-culture, target cells were labeled with CellTrace dye (Invitrogen) according to the manufacturer's protocol. TIM4 CASTL, VIII CAR, VIIIxTIM4, or nontransduced (NT) T cells were co-cultured with the designated target cell in T cell medium for 24 h in 96-well round-bottom plates. After 24 h, tumor cells were detached using trypsin and resuspended in 100 μl FACS buffer with 10 μl CountBright beads (Life Technologies Absolute Counting Beads, C36950) per well. Remaining viable tumor cells were quantified with flow cytometry. Percent lysis was calculated by counting remaining viable tumor cells in experimental wells versus tumor-only control wells, normalized as cells per bead and expressed as percent survival compared with tumor-only control wells (percent survival=((experimental well viable cells÷bead count)÷(tumor-only well viable cells÷bead count))×100.

### Animal Models

The IACUC at Duke University Medical Center approved all experimental procedures. Animal experiments involved the use of female mice at 6-12 weeks of age. Animals were maintained under pathogen-free conditions, in temperature- and humidity-controlled housing, with free access to food and water, under a 12-h light-dark cycle at the Cancer Center Isolation Facility of Duke University Medical Center. C57BL/6 mice (Charles River Laboratories) were used for syngeneic tumor implantation of CT2A. For intracranial implantation, tumor cells in PBS were mixed 1:1 with 3% methylcellulose and loaded into a 250 μL syringe (Hamilton). The needle was positioned 2 mm to the right of the bregma and 4 mm below the surface of the skull at the coronal suture using a stereotactic frame. Cells were delivered in a volume of 5 μl per mouse. For CT2A, 1×10⁴ cells were implanted.

24 hours prior to TIM4 CASTL administration, mice were treated i.p. with high dose (400 mg/kg) temozolomide (TMZ) as a method of lymphodepletion preconditioning. Unless stated otherwise, TMZ treatment occurred on day 5 post-tumor implantation and intratumoral administration of 2×10⁶ TIM4 CASTL or NT T cells occurred on day 6 post-tumor implantation. Where indicated, 4-1BB agonist antibody (clone LOB12.3; BioXCell: BE0169) was administered i.p. on days 6,9, and 12 at a dose of 200 μg per mouse.

### Results

### Phosphatidylserine is Detectable on the Surface of Tumor Cells

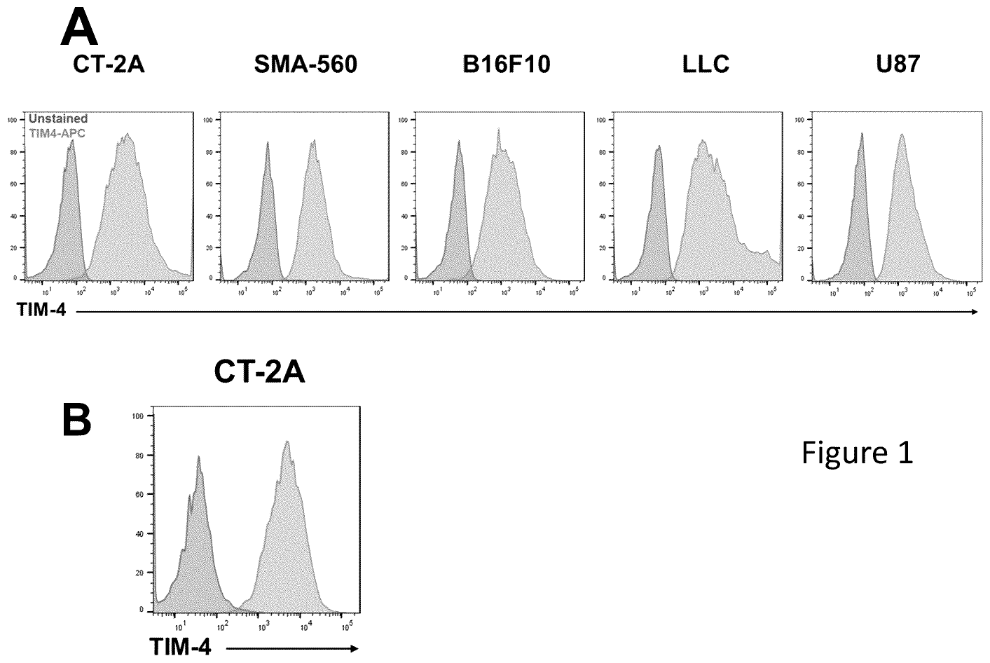

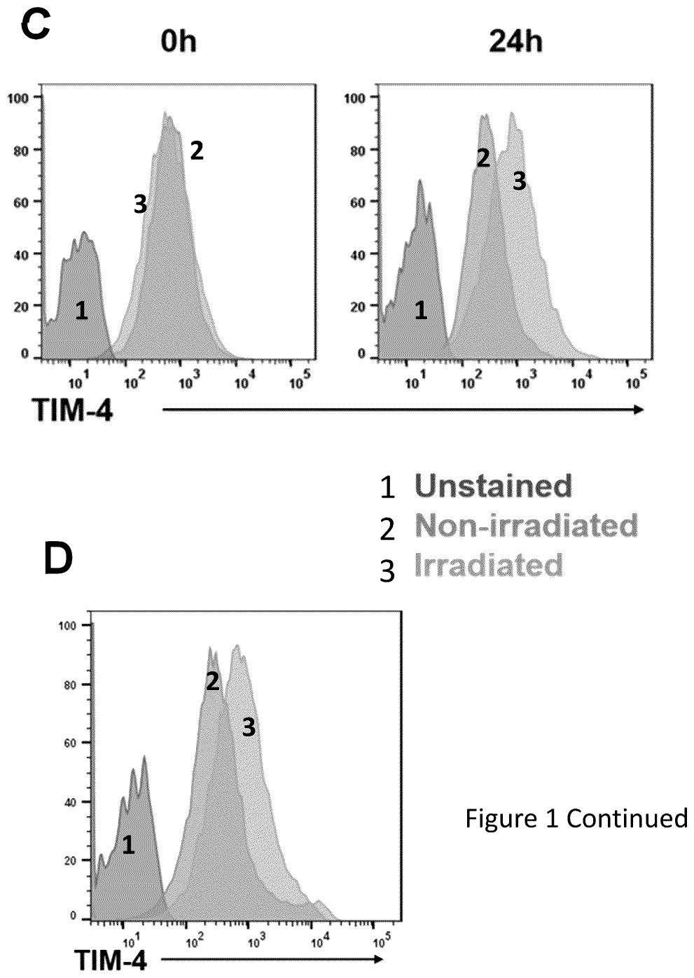

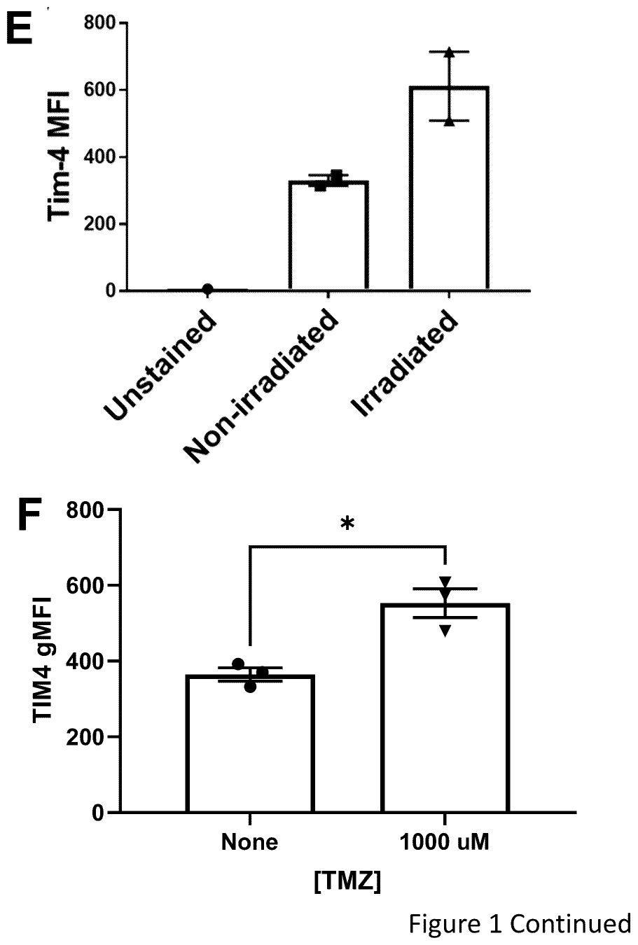

To determine if phosphatidylserine (PS) is present on tumor cells and is targetable by TIM4, we stained various tumor cell lines with recombinant human TIM4 conjugated to APC. Human TIM4 and mouse TIM4 share the same PS-binding site, so TIM4 from either species should bind PS on human or murine cells. The cell lines initially investigated included murine glioma (CT2A and SMA-560), murine melanoma (B16F10), murine lung (LLC), and human glioma (U87). Compared to unstained controls, all cell lines displayed detectable TIM4 binding, suggesting PS exposure on the surface of multiple tumor types (FIG. 1A). Importantly, these cells were viable based on Live/Dead staining. We next wanted to confirm that PS exposure and TIM4 binding was maintained in in vivo tumors. CT2A glioma was implanted intracranially (IC) in syngeneic C57BL/6 mice. After 18 days when the tumor became readily apparent, the tumor was excised and stained with TIM4-APC. Similar to in vitro cultured CT2A, ex vivo CT2A displayed appreciable TIM4 binding, further validating PS as a potential therapeutic target on tumor cells (FIG. 1B).

Enhanced PS exposure is a consequence of cellular stress. Therefore, we hypothesized that processes that promote cellular stress, like radiotherapy and chemotherapy, would further augment PS exposure on the surface of tumor cells. To test this hypothesis, in vitro cultured CT2A cells underwent exposure to 10 Gy gamma-irradiation or were left non-irradiated. 24 hours after irradiation, the cells were analyzed for PS exposure and TIM4 binding. Irradiation increased the mean fluorescence intensity (MFI) of TIM4-APC, suggesting an increase in PS exposure in the 24 hours following exposure to radiation (FIG. 1C). Next, we implanted CT2A IC in C57BL/6 and allowed the tumors to grow for 15 days. Mice were then subjected to 10 Gy total body irradiation (TBI) or left untreated. 48 hours later, tumors were excised and stained with TIM4-APC. Radiation significantly increased the MFI of TIM4 binding to tumor compared to non-irradiated tumors (FIG. 1D,E). Temozolomide (TMZ) is a chemotherapeutic agent that is part of the standard-of-care treatment regimen for GBM. Consequently, we treated CT2A cells in vitro with TMZ to determine if PS exposure was enhanced. Indeed, when CT2A was cultured in the presence of 1 mM TMZ for 24 hours, PS exposure as indicated by TIM4 binding was significantly increased (FIG. 1F). Altogether, these data suggest that PS is exposed on the surface of tumor cells, exposure that is enhanced following radiotherapy and chemotherapy. These data support the hypothesis that PS can be therapeutically targeted using TIM4 as a targeting moiety.

### TIM4 CASTL Redirects T Cells to Kill Tumor Cells

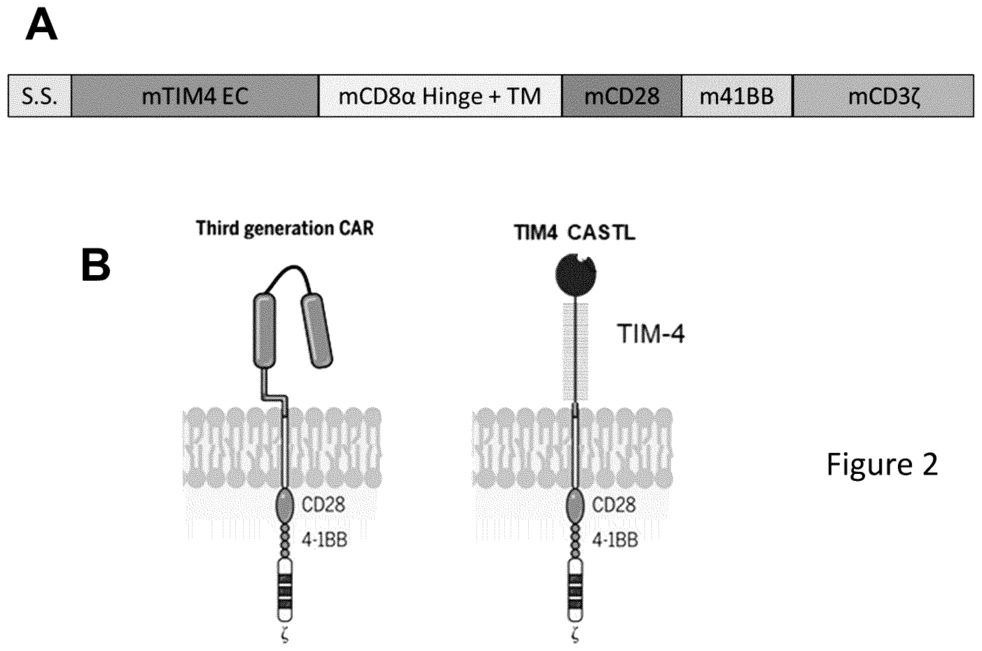

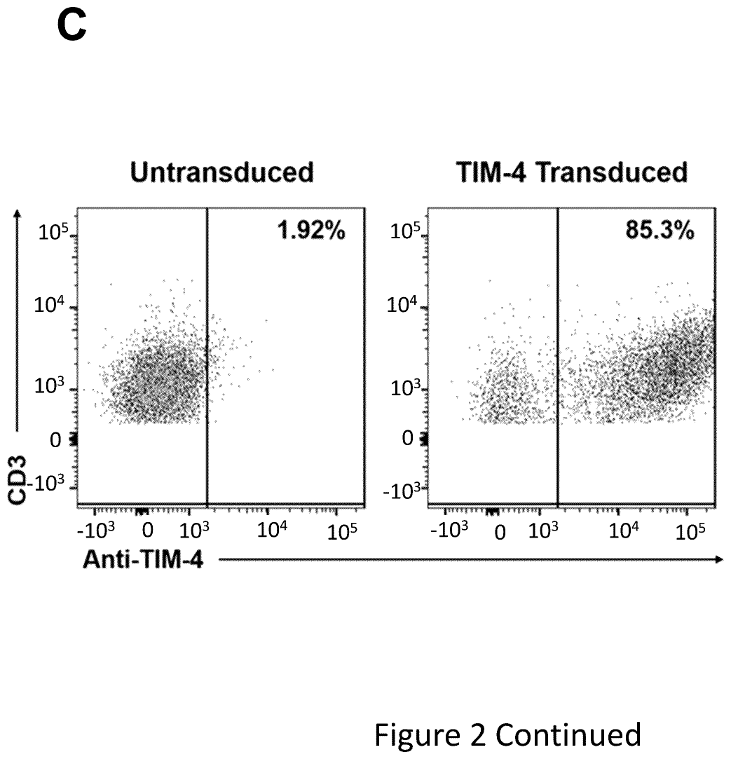

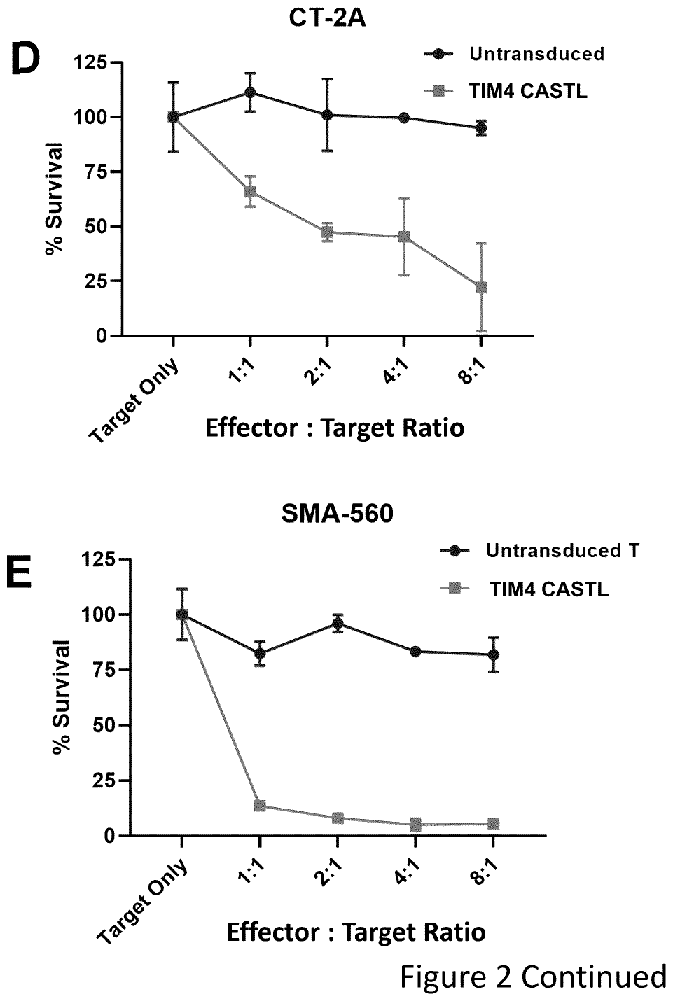

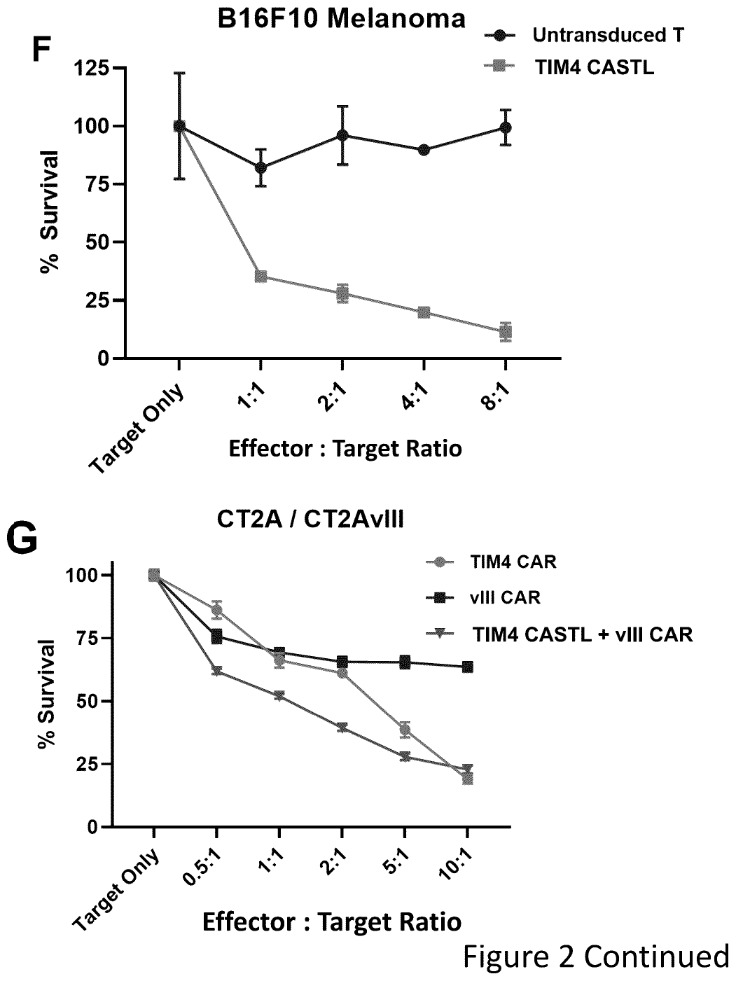

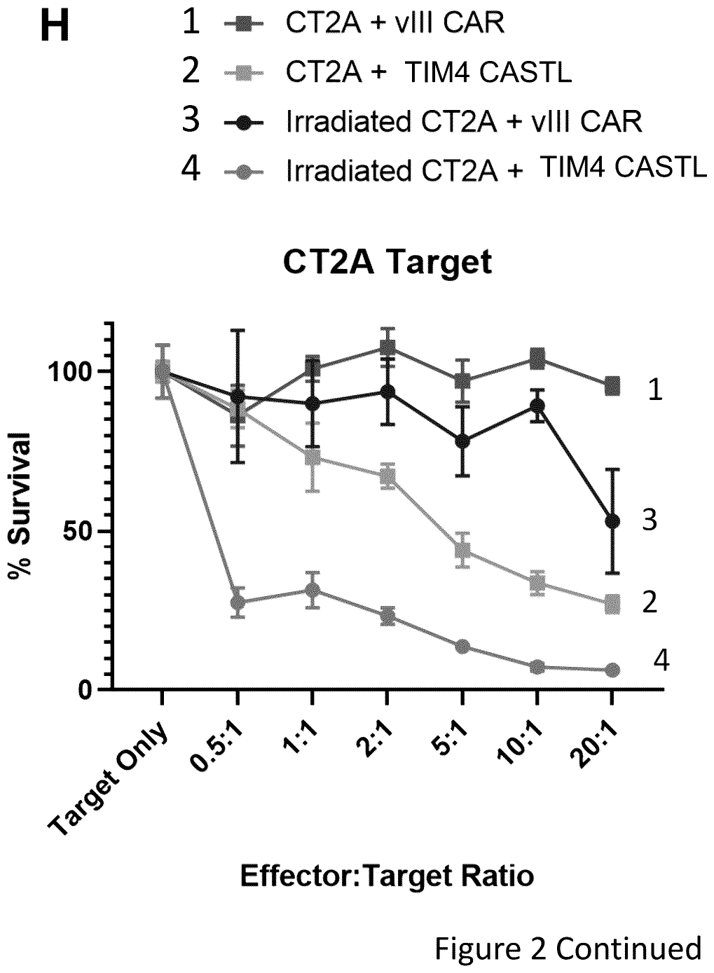

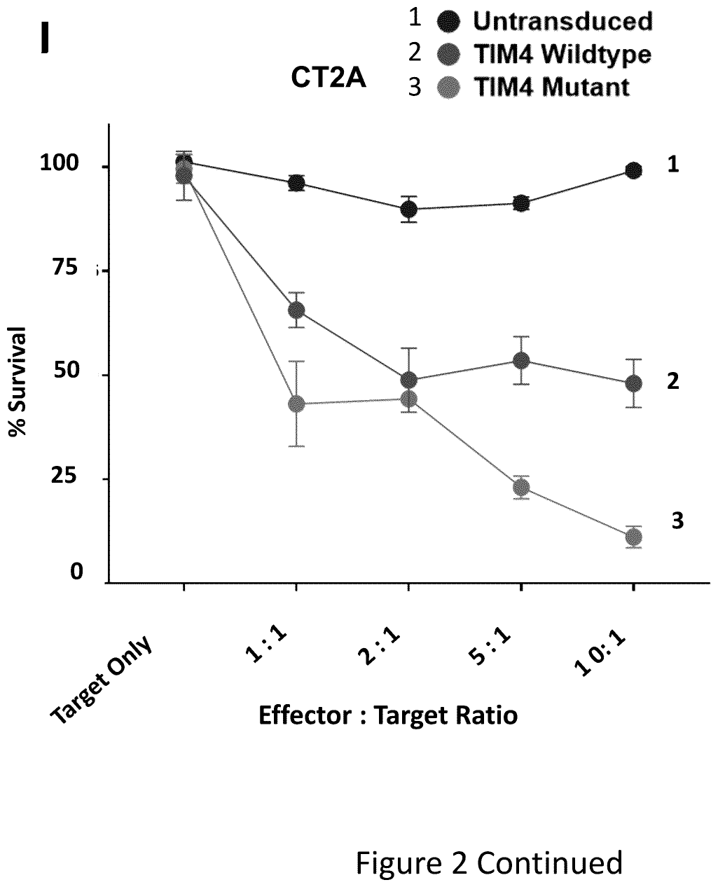

CAR T cells function by redirecting T cells to a surface antigen present on tumor cells. Typically, CAR T cells consist of a protein antigen-targeting scFv linked to intracellular T cell signaling components. CAR T cells have achieved considerable success in treating certain hematological malignancies, but their potential has yet to be realized in the context of solid tumors. We undertook a similar approach to CAR technology to target PS on tumor cells by fusing TIM4 to the T cell co-stimulatory and signaling domains of CD28, 4-1BB, and CD3ζ (FIG. 2A). As we are targeting a phospholipid, not a protein, and utilizing the natural receptor for PS, not an scFv, our approach differs from the traditional CAR platform. Thus, we termed our platform TIM4 chimeric apoptotic signal targeting lymphocyte (CASTL) (FIG. 2B). We retrovirally transduced activated mouse T cells with TIM4 CASTL and measured transduction efficiency using an anti-TIM4 antibody. Notably, T cells do not normally express TIM4. Following transduction, T cells expressed high levels of TIM4 on their surface (FIG. 2C), suggesting successful transduction of T cells with TIM4 CASTL.

Next, we sought to determine if TIM4 CASTL can redirect T cells to kill tumor cells. Untransduced T cells or TIM4 CASTL (effector) were co-cultured with CellTrace Violet (CTV)-stained CT2A cells (target) at various effector:target ratios. After 24 hours, we quantified the remaining CTV⁺ cells by flow cytometry and normalized to ‘tumor only’ conditions to generate a percent survival. TIM4 CASTL resulted in a dose-dependent cytotoxicity of CT2A, while untransduced T cells had no effect (FIG. 2D). In similar cytotoxicity assays, TIM4 CASTL potently killed SMA-560 glioma (FIG. 2E) and B16F10 melanoma (FIG. 2F), suggesting the applicability of TIM4 CASTL for non-gliomas.

The primary goal of this study was to develop an immune-based therapy that remains effective in conditions of tumor heterogeneity where traditional protein-targeting approaches fail. In GBM, the EGFR mutant variant IIII (EGFRvIII) is a tumor-specific antigen that is present in upwards of 30% of GBM patients. However, attempts to treat patients with EGFRvIII-targeting platforms, including EGFRvIII-targeting CAR T cells, have failed because, within these patients, only 30-50% of the cells within a tumor express EGFRvIII, leading to escape and outgrowth of EGFRvIII-negative cells. In contrast, we showed that PS is uniformly detectable on multiple tumor cells of different origins (FIG. 1A). We hypothesized then that TIM4 CASTL would outperform an EGFRvIII-targeting (VIII) CAR in the context of EGFRvIII heterogeneous tumors. To test this hypothesis, we co-cultured TIM4 CASTL, vIII CAR, and a combination of TIM4 CASTL and vIII CAR with the clinically relevant 70:30 mixture of CT2A:CT2AvIII for 24 hours, and then measured tumor cytotoxicity. The vIII CAR plateaued at around 75% survival; whereas, TIM4 CASTL reached 25% survival at the highest effector:target ratio (FIG. 2G). TIM4 CASTL and vIII CAR had a slight synergistic benefit as there was more robust killing at lower effector:target ratios than TIM4 CASTL or vIII CAR alone, but the maximum cytotoxicity was not different than TIM4 CASTL alone. These data provide evidence that TIM4 CASTL is more effective than vIII CAR in a clinically relevant in vitro model of tumor heterogeneity.

In line with our observation that radiation enhances PS exposure on tumor cells, we hypothesized that radiation would enhance the potency and efficacy of TIM4 CASTL. To test this hypothesis, we irradiated CT2A cells or left them non-irradiated. We then co-cultured these irradiated or non-irradiated CT2A with TIM4 CASTL or vIII CAR. TIM4 CASTL killed non-irradiated CT2A similarly to previous assays. However, TIM4 CASTL killed irradiated CT2A significantly more potently and robustly than non-irradiated cells (FIG. 2H). Importantly, vIII CAR did not substantially kill non-irradiated or irradiated CT2A. A similar effect occurred when CT2AvIII cells were used as the target population. Radiation enhanced TIM4 CASTL cytotoxicity but had no effect on vIII CAR cytotoxicity. Together, these data show that TIM4 CASTL can be successfully generated and that TIM4 CASTL can kill multiple tumor cell lines, killing that is further enhanced when coupled with radiation.

While TIM4 CASTL clearly demonstrates tumor cytotoxicity, we wanted to determine if we could enhance this activity by modifying the PS-binding site of TIM4. Three amino acid residues (WFN) are important for the metal ion-dependent binding of PS by TIM4. We hypothesized that by mutating these residues, we would decrease the affinity of PS binding, potentially leading to better tumor kill by TIM4 CASTL. While this seems counterintuitive, TIM4 CASTL can interact with low levels of PS that is present on activated T cells which exhausts or suppresses their effector function. By lowering the affinity of the interaction, only the high levels of PS that are exposed on the surface of tumor cells will trigger TIM4 CASTL activation, resulting in more specific and effective tumor kill. We engineered a mutant TIM4 CASTL by modifying the WFN binding site to a triple alanine (AAA) mutant. When assayed for in vitro tumor cytotoxicity, indeed the TIM4 mutant CASTL was more potent than the TIM4 wildtype mutant.

### TIM4 CASTL can Provide a Survival Benefit in a Model of Aggressive Glioma

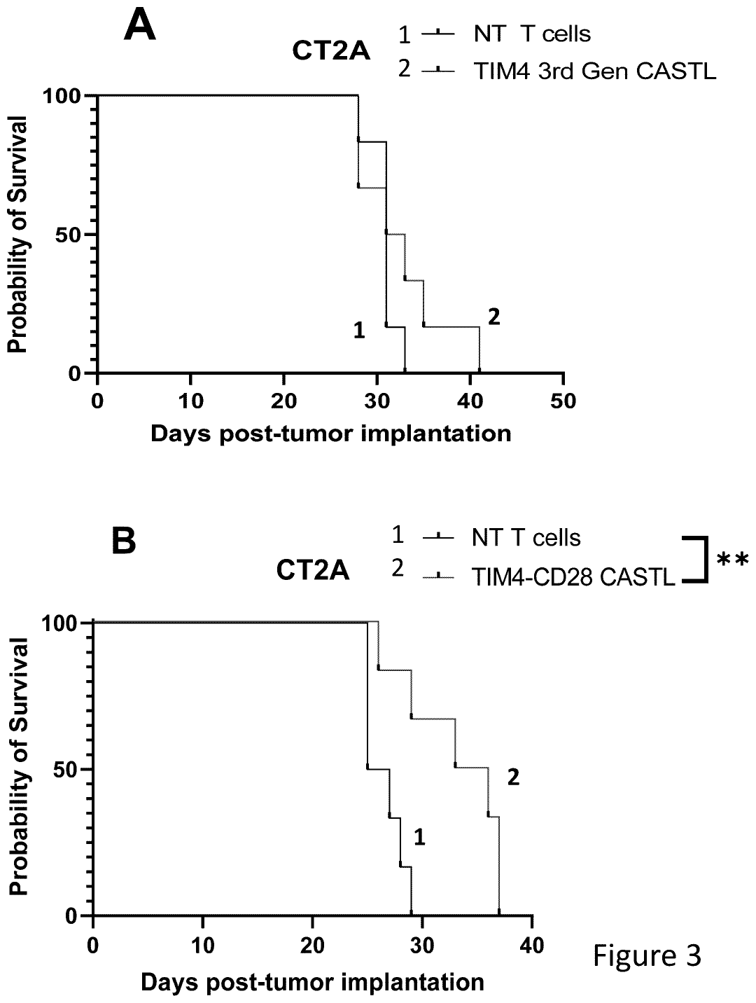

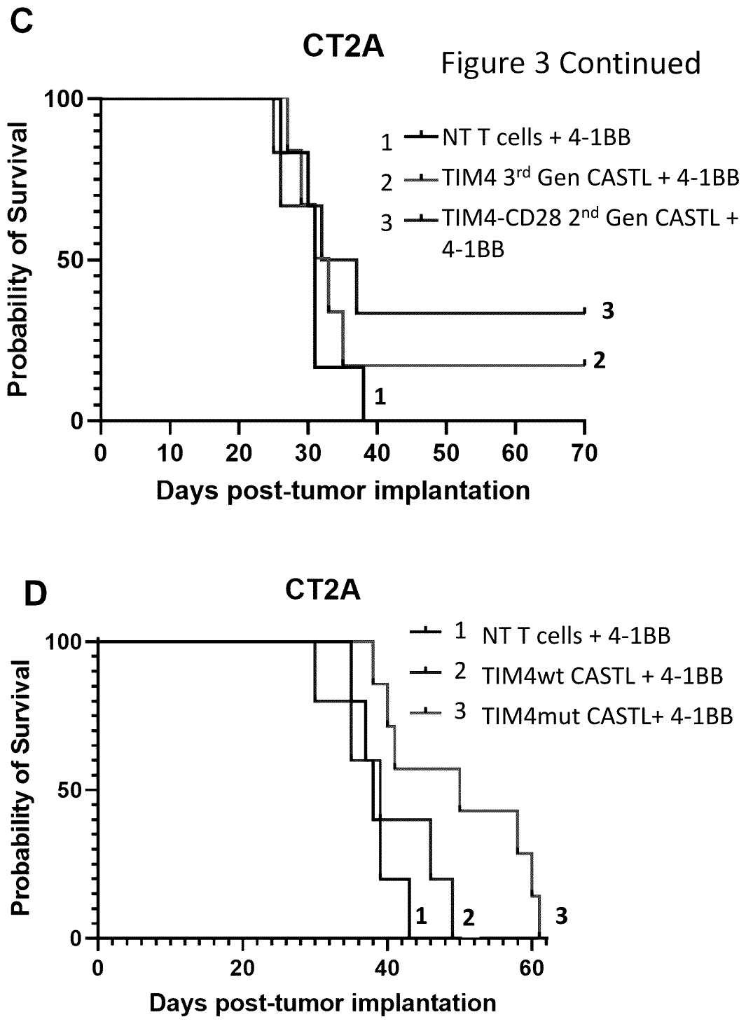

We assessed TIM4 CASTL in an immunocompetent in vivo model of CT2A murine glioma. Tumor-bearing mice underwent lymphodepletion preconditioning, a prerequisite for adoptive T cell therapeutic success, prior to TIM4 CASTL treatment. Following lymphodepletion, Nontransduced (NT) T cells or 3^{rd} generation TIM4 CASTL were administered IC at the tumor site. IC administration of TIM4 CASTL is predicted to circumvent the poor trafficking of T cells to IC tumors, while also limiting the potential for peripheral on-target, off-tumor toxicities. Initial studies with the third generation TIM4 CASTL did not yield any survival benefit (FIG. 3A). TIM4 CASTL does not rely on a traditional scFv for target binding, nor does it target a protein ligand. Thus, we hypothesized that only including a CD28 co-stimulatory domain and excluding the 4-1BB co-stimulatory domain might result in enhanced effector function of TIM4 CASTL. Indeed, a second generation TIM4 CASTL consisting only of the CD28 co-stimulatory domain and CD3ζ signaling domain (TIM4-CD28 CASTL) did provide a modest but significant survival advantage over Mock CAR T cells, with median survival being increased from 26 days to 34.5 days (FIG. 3B). Since TIM4 CASTL is cultured for several days prior to administration, we hypothesized that TIM4 CASTL is recognizing PS on dying cells or activated T cells and becoming refractory to stimulation upon in vivo administration, resulting in this modest survival benefit. Therefore, in an effort to boost TIM4 CASTL activation upon tumor recognition, we concurrently treated mice with a 4-1BB agonist. While the median survival of mice treated with either a third generation or second generation TIM4 CASTL plus 4-1BB was not significantly improved, 20% and 40% of mice survived long-term, respectively (FIG. 3C). Together, these data suggest that a 2^{nd} generation TIM4-CD28 CASTL has moderate in vivo activity which can potentially be further enhanced with T cell activating immunotherapy.

Next, we tested TIM4 mutant in vivo in combination with 4-1BB agonism. Compared to mice receiving treatment with wildtype TIM4 (TIM4wt), mice receiving mutant TIM4 (TIM4mut) CASTL survived significantly longer (FIG. 3D). These data, along with data from FIG. 2I, suggest that altering the interaction between TIM4 and PS can enhance the efficacy of TIM4 CASTL.

### SEQUENCES

An exemplary human TIM-4 CASTL sequence is shown below. One skilled in the art will readily appreciate that the sequence can be modified with known alternative signal sequences, hinge and transmembrane sequences as well as various combinations of co-stimulatory domains which may be included in any order. For example, human a TIM-4 CASTL of SEQ ID NOs: 5 and 6 do not comprise a 4-1BB co-stimulatory domain. Additionally, species specific sequences and sequence variants may be used. For example, human mutant TIM-4 CASTL of SEQ ID NOs: 4 and 6 wherein the WFN residues are mutated to AAA residues, or SEQ ID NOs: 1 and 2, which are murine TIM-4 CASTL fusion proteins.

Human TIM-4 CASTL
SEQ ID NO: 3
MVLLVTSLLLCELPHPAFLLIPETVVTEVLGHRVTLPCLYSSWSHNSNSMCWGKDQCP
YSGCKEALIRTDGMRVTSRKSAKYRLQGTIPRGDVSLTILNPSESDSGVYCCRIEVP
G [symbol] DVKINVRLNLQRASTTTHRTATTTTRRTTTTSPTTTRQMTTTPAALPTTVV
TTPDLTTGTPLQMTTIAVFTTANTCLSLTPSTLPEEATGLLTPEPSKEGPILTAESET
VLPSDSWSSVESTSADTVLLTSKESKVWDLPSTSHVSMWKTSDSVSSPQPGASDTAV
PEQNKTTKTGQMDGIPMSMKNEMPISQAAFVPVFLPAKPTTTPAPRPPTPAPTIASQPL

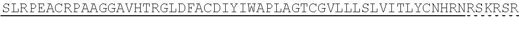

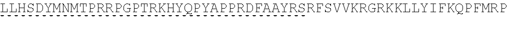

VQTTQEEDGCSCRFPEEEEGGCELRVKFSRSADAPAYQQGQNQLYNELNLGRREEYDV
LDKRRGRDPEMGGKPRRKNPQEGLYNELQKDKMAEAYSEIGMKGERRRGKGHDGLY
QGLSTATKDTYDALHMQALPPR
Signal Sequence
(SEQ ID NO: 9)
GMCSFR
Human TIM-4 Extracellular Domain
(SEQ ID NO: 7)
with mutated residues in large font
Human CD8 Hinge and Transmembrane
(SEQ ID NO: 10)
Human CD28
(SEQ ID NO: 11)
Human 4-1BB
(SEQ ID NO: 12)
Human CD3zeta
(SEQ ID NO: 13)

What is claimed:

1. A TIM-4 fusion protein comprising a TIM-4 extracellular domain and at least one co-stimulatory domain, wherein the TIM-4 extracellular domain comprises SEQ ID NO: 7 or a sequence having at least 95% identity to SEQ ID NO: 7.

2. The TIM-4 fusion protein of claim 1, wherein the TIM-4 extracellular domain comprises SEQ ID NO: 8.

3. The fusion protein of claim 1, wherein the co-stimulatory domain comprises CD3 co-stimulatory domain.

4. The fusion protein of claim 3, wherein the co-stimulatory domain further comprises one or both of a co-stimulatory domain from CD28 and 4-1BB.

5. The fusion protein of claim 4, wherein the fusion protein further comprises a signal sequence and a transmembrane domain.

6. The fusion protein of claim 5, wherein the fusion protein comprises SEQ ID NO: 3 or a sequence having at least 95% identity to SEQ ID NO: 3.

7. The fusion protein of claim 5, wherein the fusion protein comprises SEQ ID NO: 5 or a sequence having at least 95% identity to SEQ ID NO: 5.

8. A construct comprising a polynucleotide sequence encoding the TIM-4 fusion protein of claim 1 operably linked to a promoter.

9. The construct of claim 8, wherein the construct is included in a lentiviral, retroviral or AAV vector.

10. A T cell comprising the construct of claim 9.

11. The T cell of claim 10, further comprising a chimeric antigen receptor (CAR).

12. The T cell of claim 11, wherein the CAR is specific for a tumor antigen.

13. The T cell of claim 12, wherein the CAR comprises an extracellular domain comprising an antigen binding region which binds to both a wildtype EGFR and an EGFR VIII variant.

14. A method of treating cancer in a subject, the method comprising administering a therapeutically effective amount of the T cell of claim 10 and a pharmaceutically acceptable excipient, carrier and/or diluent.

15. The method 14, wherein the cancer is a PS-associated cancer.

16. The method of claim 14, wherein the T cell is administered intratumorally.

17. The method of claim 14, wherein the subject is administered radiation and/or chemotherapy prior to administration of the T cell.

18. The method of claim 14, further comprising administering a T cell activating immunotherapy.

19. A method of treating cancer, the method comprising administering a therapeutically effective amount of the T cell of claim 12 and a pharmaceutically acceptable excipient, carrier and/or diluent.

20. The method of claim 19, wherein the cancer is an EGFR-associated cancer.

21. The method of claim 20, wherein the EGFR-associated cancer comprises a glioma, glioblastoma, medulloblastoma, ependymoma, diffuse intrinsic pontine glioma (DIPG), a brain metastases, head and neck, ovarian, cervical, bladder or esophageal cancer.

22. The method of claim 19, wherein the T cell is administered intratumorally or systemically.

23. The method of claim 19, wherein the subject is administered radiation and/or chemotherapy prior to administration of the T cell.

24. The method of claim 19, further comprising administering a T cell activating immunotherapy.


SEQUENCE LISTING (13 sequences)

SEQ ID NO: 1 - protein, 555 aa, synthetic construct
  REGION 1..22: note = GMCSFR Signal Sequence
  REGION 23..281: note = GMCSFR Signal Sequence 1-22 Mouse TIM4 Extracellular Domain
  REGION 285..356: note = Mouse CD8 Hinge and Transmembrane
  REGION 357..397: note = Mouse CD28
  REGION 398..442: note = Mouse 4-1BB
  REGION 443..555: note = Mouse CD3zeta
  REGION 1..555: note = Murine TIM4 CASTL
MVLLVTSLLLCELPHPAFLLIPPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTAAATTTKPVLRTPSPVHPTGTSQPQRPEDCRPRGSVKGTGLDFACDIYIWAPLAGICVALLLSLIITLICYHRSRNSRRNRLLQVTTMNMTPRRPGLTRKPYQPYAPARDFAAYRPKWIRKKFPHIFKQPFKKTTGAAQEEDACSCRCPQEEEGGGGGYELRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 2 - protein, 555 aa, synthetic construct
  COMPBIAS 1..555: note = Murine TIM4 mutant CASTL
  MUTAGEN 121..123: note = PS-binding site Mutation
MVLLVTSLLLCELPHPAFLLIPPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGAAADVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTAAATTTKPVLRTPSPVHPTGTSQPQRPEDCRPRGSVKGTGLDFACDIYIWAPLAGICVALLLSLIITLICYHRSRNSRRNRLLQVTTMNMTPRRPGLTRKPYQPYAPARDFAAYRPKWIRKKFPHIFKQPFKKTTGAAQEEDACSCRCPQEEEGGGGGYELRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 3 - protein, 597 aa, synthetic construct
  REGION 1..597: note = Human TIM4 CASTL
  REGION 1..22: note = GMCSFR Signal Sequence
  REGION 23..312: note = Human TIM4 Extracellular Domain
  REGION 315..397: note = Human CD8 Hinge and Transmembrane
  REGION 398..438: note = Human CD28
  REGION 439..485: note = Human 4-1BB
  REGION 486..597: note = Human CD3zeta
MVLLVTSLLLCELPHPAFLLIPETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 4 - protein, 597 aa, synthetic construct
  REGION 1..597: note = Human mutant TIM4 CASTL
  MUTAGEN 117..119: note = PS-binding site Mutation
MVLLVTSLLLCELPHPAFLLIPETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGAAADVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 5 - protein, 550 aa, synthetic construct
  REGION 1..550: note = Human TIM4 CASTL 2nd gen
  REGION 1..22: note = GMCSFR Signal Sequence
  REGION 23..312: note = Human TIM4 Extracellular Domain
  REGION 315..397: note = Human CD8 Hinge and Transmembrane
  REGION 398..438: note = Human CD28
  REGION 439..550: note = Human CD3zeta
MVLLVTSLLLCELPHPAFLLIPETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 6 - protein, 550 aa, synthetic construct
  REGION 1..550: note = Human mutant TIM4 CASTL 2nd gen
  MUTAGEN 117..119: note = PS-binding site Mutation
MVLLVTSLLLCELPHPAFLLIPETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGAAADVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 7 - protein, 290 aa, synthetic construct [sequence omitted]

SEQ ID NO: 8 - protein, 290 aa, synthetic construct
  MUTAGEN 95..97: note = Human TIM4 Extracellular Domain mutated residues
ETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGAAADVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQ

SEQ ID NO: 9 - protein, 22 aa, synthetic construct
MVLLVTSLLLCELPHPAFLLIP

SEQ ID NO: 10 - protein, 83 aa, synthetic construct
FVPVFLPAKPTTTPAPRPPTPAPTIASQPLSLRPEACRPAAGGAVHTRGLDFACDIYIWAPLAGTCGVLLLSLVITLYCNHRN

SEQ ID NO: 11 - protein, 41 aa, synthetic construct
RSKRSRLLHSDYMNMTPRRPGPTRKHYQPYAPPRDFAAYRS

SEQ ID NO: 12 - protein, 47 aa, synthetic construct
RFSVVKRGRKKLLYIFKQPFMRPVQTTQEEDGCSCRFPEEEEGGCEL

SEQ ID NO: 13 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR